(19) United States
(12) Patent Application Publication
(10) Pub. No.: US 2024/0198063 A1
(43) Pub. Date: Jun. 20, 2024

(54) SYSTEMS AND METHODS FOR IMPROVING CEREBROSPINAL FLUID (CSF) DRAINAGE

(71) Applicant: THE METHODIST HOSPITAL SYSTEM, Houston, TX (US)

(72) Inventors: Eugene V. GOLANOV, Houston, TX (US); Angelique S. REGNIER-GOLANOV, Houston, TX (US); Christof KARMONIK, Houston, TX (US); Gavin W. BRITZ, Houston, TX (US); Alan David ESKURI, Hanover, MN (US)

(21) Appl. No.: 18/288,019

(22) PCT Filed: Apr. 21, 2022
(86) PCT No.: PCT/US2022/025703

§ 371 Date: Oct. 23, 2023

Related U.S. Application Data
(60) Provisional application No. 63/178,616, filed on Apr. 23, 2021.

(51) Int. Cl. A61M 27/00; A61N 1/04; A61N 1/05; A61N 1/36

(52) U.S. Cl. CPC A61M 27/006; A61N 1/36003; A61N 1/36031; A61N 1/36034; A61N 1/36135; A61N 1/36178; A61M 2205/054; A61N 1/0452; A61N 1/0551

(57) ABSTRACT

Systems and methods for draining cerebrospinal fluid (CSF) are described herein described herein. In one implementation, an example system includes a signal generator, a plurality of electrodes operably connected to the signal generator, and a controller operably connected to the signal generator. The controller includes a processor and a memory. The controller is configured to deliver a neuromuscular electrical stimulation signal including at least one burst of pulses to at least one muscle in the subject's neck. The neuromuscular electrical stimulation signal is configured to induce a plurality of contractions of the at least one muscle. Additionally, the contractions of the at least one muscle are configured to squeeze at least one lymph node to create a pumping force, and the pumping force is configured to direct CSF flow in a proximal direction. The results in CSF drainage through the subject's neck lymphatic system.

### CROSS-REFERENCE TO RELATED APPLICATIONS

This application claims the benefit of U.S. provisional patent application No. 63/178,616, filed on Apr. 23, 2021, and titled “SYSTEMS AND METHODS FOR IMPROVING CEREBROSPINAL FLUID (CSF) DRAINAGE,” the disclosure of which is expressly incorporated herein by reference in its entirety.

### BACKGROUND

Cerebrospinal fluid (CSF), which fills the cerebral ventricles and subarachnoid space surrounding the brain and the spinal cord, serves numerous purposes vital for the normal brain function. It provides mechanical support, trophic function and maintenance of normal biochemical environment. An important function of CSF is the clearance of the brain metabolic waste and various pathogenic elements such as abnormal proteins.

Thus, maintenance of normal CSF drainage becomes of utmost importance for the normal brain and abnormalities of CSF drainage are being linked to neurodegenerative diseases, such as Alzheimer's disease, multiple sclerosis, traumatic brain injury, subarachnoid and intracerebral hemorrhages, stroke, hydrocephalus, and microgravity related hydrocephalus. Therefore, normalization or improvement of an unsatisfactory CSF drainage will exert positive effect on these and other conditions.

### SUMMARY

Systems and methods for improving CSF drainage are described herein. It is known that 50% of CSF is drained through the neck lymphatic vessels and nodes. Accordingly, the systems and methods described herein stimulate lymph movement through the neck lymphatic system, which accelerates the drainage of CSF, improves its circulation throughout the subarachnoid space, and accelerates the clearance of wasteful and pathological materials. The systems and methods described herein can therefore alleviate pathological conditions that benefit from the improved CSF drainage. Such conditions include, but are not limited to, Alzheimer's disease, subarachnoid hemorrhage, traumatic brain injury, intracerebral hemorrhage, multiple sclerosis, and hydrocephalus (normal pressure, microgravity-induced).

An example system for draining cerebrospinal fluid (CSF) though a subject's neck lymphatic system is described herein. The system includes a signal generator, a plurality of electrodes operably connected to the signal generator, and a controller operably connected to the signal generator. The controller includes a processor and a memory, and the memory has computer-executable instructions stored thereon. The controller is configured to deliver a neuromuscular electrical stimulation signal including at least one burst of pulses to at least one muscle in the subject's neck. The neuromuscular electrical stimulation signal is configured to induce a plurality of contractions of the at least one muscle. Additionally, the contractions of the at least one muscle are configured to squeeze at least one lymph node to create a pumping force, and the pumping force is configured to direct CSF flow in a proximal direction.

In some implementations, the at least one burst of pulses includes rectangular pulses. Optionally, the rectangular pulses are monophasic or biphasic pulses. Alternatively or additionally, the rectangular pulses have a duration of about 0.5-1,000 microseconds (usec).

In some implementations, the at least one burst of pulses includes sinusoidal pulses. Optionally, the sinusoidal pulses have a frequency of about 5-150 Hertz (Hz).

Alternatively or additionally, in some implementations, the neuromuscular electrical stimulation signal is delivered with a current of about 10-100 milliamps (mA).

In some implementations, the system optionally includes a sensor configured to detect muscular contractions. For example, the sensor is optionally a strain gauge. The controller is further configured to receive a feedback signal from the sensor. The feedback signal includes information related to a contraction state of the at least one muscle. The controller can be further configured to increase a magnitude of the neuromuscular electrical stimulation signal current until detecting a contraction of the at least one muscle. The controller can be further configured to increase the magnitude of the neuromuscular electrical stimulation signal current above a minimum current that induces the contraction of the at least one muscle. For example, the magnitude of the neuromuscular electrical stimulation signal current can be increased about 10-50% above the minimum current.

Alternatively or additionally, in some implementations, the neuromuscular electrical stimulation signal includes a plurality of bursts of pulses. The plurality of bursts of pulses can be a series of about 5-50 bursts of pulses. Optionally, the series is delivered at a frequency of about 0.02-1 Hz.

Alternatively or additionally, in some implementations, the neuromuscular electrical stimulation signal is delivered for a period of about 5-60 minutes.

Alternatively or additionally, in some implementations, the at least one muscle is one or more of musculus platysma, sternocleidomastoid, or trapezius.

Alternatively or additionally, in some implementations, the plurality of electrodes are surface electrodes. Alternatively, in other implementations, the plurality of electrodes are implantable electrodes. Additionally, the signal generator is optionally an implantable signal generator.

An example method for draining cerebrospinal fluid (CSF) though a subject's neck lymphatic system is also described herein. The method includes positioning a plurality of electrodes in proximity to at least one muscle in the subject's neck; and delivering, using the plurality of electrodes, a neuromuscular electrical stimulation signal that includes at least one burst of pulses to the at least one muscle. The method also includes inducing, using the neuromuscular electrical stimulation signal, a plurality of contractions of the at least one muscle; and creating a pumping force in at least one lymph node using the plurality of contractions of the at least one muscle. The pumping force directs CSF flow in a proximal direction.

In some implementations, the method optionally includes treating a disease or condition in the subject by directing CSF flow in the proximal direction. For example, the disease or condition is hydrocephalus, a neurodegenerative disorder, or a sleep disorder.

Alternatively or additionally, in some implementations, the method optionally includes improving the subject's sleep health by directing CSF flow in the proximal direction.

Alternatively or additionally, in some implementations, the method optionally includes improving circulation of neurotrophic agents by directing CSF flow in the proximal direction.

Alternatively or additionally, in some implementations, the method optionally includes improving removal of biological waste agents by directing CSF flow in the proximal direction.

In some implementations, the at least one burst of pulses includes rectangular pulses.

In some implementations, the at least one burst of pulses includes sinusoidal pulses.

Alternatively or additionally, in some implementations, the neuromuscular electrical stimulation signal is delivered with a current of about 10-100 milliamps (mA).

Alternatively or additionally, in some implementations, the method optionally includes receiving, from a sensor configured to detect muscular contractions, a feedback signal, wherein the feedback signal comprises information related to a contraction state of the at least one muscle. The method can include increasing a magnitude of the neuromuscular electrical stimulation signal current until detecting a contraction of the at least one muscle. The method can further include increasing the magnitude of the neuromuscular electrical stimulation signal current above a minimum current that induces the contraction of the at least one muscle. For example, the magnitude of the neuromuscular electrical stimulation signal current can be increased about 10-50% above the minimum current.

Alternatively or additionally, in some implementations, the neuromuscular electrical stimulation signal includes a plurality of bursts of pulses.

Alternatively or additionally, in some implementations, the neuromuscular electrical stimulation signal is delivered for a period of about 5-60 minutes.

Alternatively or additionally, in some implementations, the at least one muscle is one or more of musculus platysma, sternocleidomastoid, or trapezius.

Another example system for draining cerebrospinal fluid (CSF) though a subject's neck lymphatic system is described herein. The system includes a plurality of mechanical actuators; and a controller operably connected to the plurality of mechanical actuators. The controller includes a processor and a memory, and the memory has computer-executable instructions stored thereon. The controller is configured to sequentially activate the mechanical actuators to exert a positive pressure sequence in proximity to at least one lymph node in the subject's neck. The at least one lymph node is located proximally with respect to the subject's carotid artery bifurcation. The positive pressure sequence is configured to squeeze the at least one lymph node to create a pumping force, and the pumping force is configured to direct CSF flow in a proximal direction.

In some implementations, the mechanical actuators are activated sequentially in the proximal direction.

Alternatively or additionally, in some implementations, each of the mechanical actuators includes an inflatable member. Optionally, an internal pressure of the inflatable member is regulated between about 2-10 millimeters of mercury (mmHg) during a mechanical activation cycle. Alternatively or additionally, a duration of the mechanical activation cycle is optionally about 500 milliseconds (msec)−5 seconds.

Alternatively or additionally, in some implementations, the mechanical actuators are activated repeatedly to exert a series of positive pressure sequences in proximity to the at least one lymph node.

Another method for draining cerebrospinal fluid (CSF) though a subject's neck lymphatic system is described herein. The method includes positioning a plurality of mechanical actuators in proximity to at least lymph node in the subject's neck, where the at least one lymph node is located proximally with respect to the subject's carotid artery bifurcation. The method also includes sequentially activating the mechanical actuators to exert a positive pressure sequence in proximity to the at least one lymph node; and creating a pumping force in at least one lymph node with the positive pressure sequence exerted in proximity to the at least one lymph node, where the pumping force directs CSF flow in a proximal direction.

In some implementations, the method optionally includes treating a disease or condition in the subject by directing CSF flow in the proximal direction. For example, the disease or condition is hydrocephalus, a neurodegenerative disorder, or a sleep disorder.

Alternatively or additionally, in some implementations, the method optionally includes improving the subject's sleep health by directing CSF flow in the proximal direction.

Alternatively or additionally, in some implementations, the method optionally includes improving circulation of neurotrophic agents by directing CSF flow in the proximal direction.

Alternatively or additionally, in some implementations, the method optionally includes improving removal of biological waste agents by directing CSF flow in the proximal direction.

In some implementations, the mechanical actuators are activated sequentially in the proximal direction.

Alternatively or additionally, in some implementations, each of the mechanical actuators includes an inflatable member. Optionally, an internal pressure of the inflatable member is regulated between about 2-10 millimeters of mercury (mmHg) during a mechanical activation cycle. Alternatively or additionally, a duration of the mechanical activation cycle is optionally about 500 milliseconds (msec)-5 seconds.

Alternatively or additionally, in some implementations, the mechanical actuators are activated repeatedly to exert a series of positive pressure sequences in proximity to the at least one lymph node.

Yet another system for draining cerebrospinal fluid (CSF) though a subject's neck lymphatic system is described herein. The system includes a vacuum suction device comprising at least one inflatable member; and a controller operably connected to the vacuum suction device. The controller includes a processor and a memory, and the memory has computer-executable instructions stored thereon. The controller is configured to activate the vacuum suction device to exert a negative pressure sequence in proximity to at least one lymph node in the subject's neck. A vacuum of the vacuum suction device is regulated between about 3-5 millimeters of mercury (mmHg) during a vacuum activation cycle. The at least one lymph node is located proximally with respect to the subject's carotid artery bifurcation. The negative pressure sequence is configured to create a suction in the at least one lymph node, and the suction is configured to direct CSF flow in a proximal direction.

In some implementations, a duration of the vacuum activation cycle is about 500 milliseconds (msec)−2 seconds.

Alternatively or additionally, in some implementations, the vacuum suction device is activated repeatedly to exert a series of negative pressure sequences in proximity to the at least one lymph node.

Yet another method for draining cerebrospinal fluid (CSF) though a subject's neck lymphatic system is described herein. The method includes positioning a vacuum suction device in proximity to at least lymph node in the subject's neck, where the at least one lymph node is located proximally with respect to the subject's carotid artery bifurcation. A vacuum of the vacuum suction device is regulated between about 3-5 millimeters of mercury (mmHg) during a vacuum activation cycle. The method also include activating the vacuum suction device to exert a negative pressure sequence in proximity to the at least one lymph node; and creating a suction in the at least one lymph node with the negative pressure sequence exerted in proximity to the at least one lymph node, and the suction directs CSF flow in a proximal direction.

In some implementations, the method optionally includes treating a disease or condition in the subject by directing CSF flow in the proximal direction. For example, the disease or condition is hydrocephalus, a neurodegenerative disorder, or a sleep disorder.

Alternatively or additionally, in some implementations, the method optionally includes improving the subject's sleep health by directing CSF flow in the proximal direction.

Alternatively or additionally, in some implementations, the method optionally includes improving circulation of neurotrophic agents by directing CSF flow in the proximal direction.

Alternatively or additionally, in some implementations, the method optionally includes improving removal of biological waste agents by directing CSF flow in the proximal direction.

In some implementations, a duration of the vacuum activation cycle is about 500 milliseconds (msec)−2 seconds.

Alternatively or additionally, in some implementations, the vacuum suction device is activated repeatedly to exert a series of negative pressure sequences in proximity to the at least one lymph node.

Yet another system for draining cerebrospinal fluid (CSF) though a subject's neck lymphatic system is described herein. The system includes an implantable balloon; an actuator fluidly connected to the implantable balloon; and a controller operably connected to the actuator. The controller includes a processor and a memory, and the memory has computer-executable instructions stored thereon. The controller is configured to inflate, using the actuator, the implantable balloon to compress the subject's cisterna magna. The implantable balloon is located at a junction of the subject's cisterna magna and perispinal subarachnoid space, and the compression is configured to direct CSF flow toward the subject's perispinal subarachnoid space.

In some implementations, the implantable balloon has a trapezoidal shape, and the implantable balloon has a pair of parallel bases and a pair of legs extending between the pair of parallel bases. Optionally, a first base of the pair of parallel bases having a longer length is thicker than a second base of the pair of parallel bases having a shorter length. The first base of the pair of parallel bases is implanted toward the subject's cerebellum.

Alternatively or additionally, in some implementations, an internal pressure of the implantable balloon is regulated between about 0-15 millimeters of mercury (mmHg) during inflation. Optionally, a rate of inflation is about 1 mmHg per second.

Yet another method for draining cerebrospinal fluid (CSF) though a subject's neck lymphatic system is described herein. The method includes placing an implantable balloon at a junction of the subject's cisterna magna and perispinal subarachnoid space; and inflating the implantable balloon to compress the subject's cisterna magna. The compression is configured to direct CSF flow toward the subject's perispinal subarachnoid space.

In some implementations, the method optionally includes treating a disease or condition in the subject by directing CSF flow in the proximal direction. For example, the disease or condition is hydrocephalus, a neurodegenerative disorder, or a sleep disorder.

Alternatively or additionally, in some implementations, the method optionally includes improving the subject's sleep health by directing CSF flow in the proximal direction.

Alternatively or additionally, in some implementations, the method optionally includes improving circulation of neurotrophic agents by directing CSF flow in the proximal direction.

Alternatively or additionally, in some implementations, the method optionally includes improving removal of biological waste agents by directing CSF flow in the proximal direction.

In some implementations, the implantable balloon has a trapezoidal shape, and the implantable balloon has a pair of parallel bases and a pair of legs extending between the pair of parallel bases. Optionally, a first base of the pair of parallel bases having a longer length is thicker than a second base of the pair of parallel bases having a shorter length. The first base of the pair of parallel bases is implanted toward the subject's cerebellum.

Alternatively or additionally, in some implementations, an internal pressure of the implantable balloon is regulated between about 0-15 millimeters of mercury (mmHg) during inflation. Optionally, a rate of inflation is about 1 mmHg per second.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram of an example system for draining CSF though a subject's neck lymphatic system according to an implementations described herein.

FIG. 2A illustrates neck muscle and lymph node arrangement in a subject. FIG. 2B illustrates example surface electrode placement relative to the neck muscle and lymph nodes of FIG. 2A.

FIG. 3 is a flow diagram illustrating example operations for draining CSF though a subject's neck lymphatic system according to an implementation described herein.

FIG. 4 is a block diagram of another example system for draining CSF though a subject's neck lymphatic system according to an implementations described herein.

FIG. 5 illustrates an example collar including a plurality of mechanical actuators (inflatable members) according to an implementation described herein.

FIG. 6 is a flow diagram illustrating example operations for draining CSF though a subject's neck lymphatic system according to another implementation described herein.

FIG. 7 is a block diagram of yet another example system for draining CSF though a subject's neck lymphatic system according to an implementations described herein.

FIG. 8 illustrates a vacuum suction device including an inflatable member according to an implementation described herein.

FIG. 9 is a flow diagram illustrating example operations for draining CSF though a subject's neck lymphatic system according to another implementation described herein.

FIG. 10 is a block diagram of yet another example system for draining CSF though a subject's neck lymphatic system according to an implementations described herein.

FIG. 11 illustrates an implantable balloon and placement in a subject according to an implementation described herein.

FIGS. 12A-12B illustrate an implantable balloon having a trapezoidal shape according to an implementation described herein. FIG. 12A shows the implantable balloon in the deflated state. FIG. 12B shows the implantable balloon in the inflated state.

FIG. 13 is a flow diagram illustrating example operations for draining CSF though a subject's neck lymphatic system according to another implementation described herein.

FIG. 14 is an example computing device.

FIG. 15A is an illustration of the lymphatic system of the head and neck. FIG. 15B is a diagram of lymph nodes levels in the neck.

FIGS. 16A-16D illustrate accelerated CSF flow in response to NMES stimulation.

FIG. 16A is a schematic showing the time lapse of contrast-enhanced magnetic resonance imaging (MRI) experiments with the cisterna magna injection. FIG. 16B shows quantification of gadolinium signal in the olfactory bulb. FIG. 16C is a representative MRI coronal section for Sham mice (i.e., no stimulation) 60 minutes after the first scan. FIG. 16D is a representative MRI coronal section for Stimulated mice (0.2 Hz_5 Hz-Bottom) 60 minutes after the first scan.

FIG. 17 illustrates an example system for draining CSF though a subject's neck lymphatic system using an implantable signal generator and implantable electrodes according to an implementation described herein.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. As used herein, the terms “about” or “approximately”, when used in reference to a linear dimension (length, width, thickness, etc.), an internal pressure, a length of time, a frequency, mean within plus or minus 10% percentage of the referenced value.

### Lymph Nodes of the Head and Neck

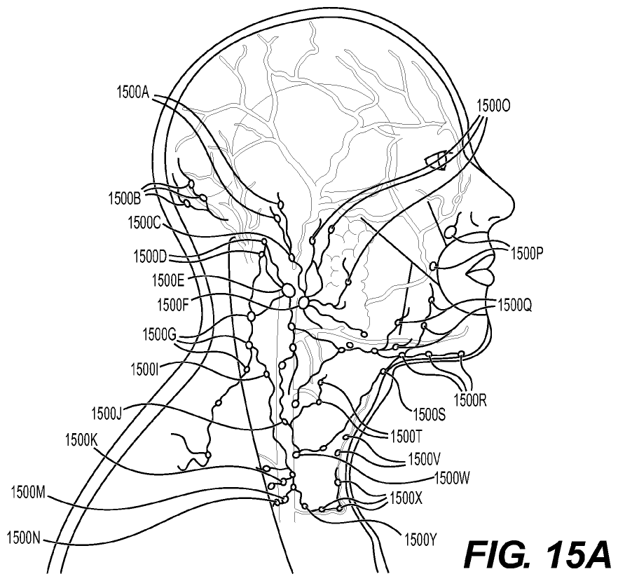

In some implementations described herein, CSF drainage is improved by non-invasively stimulating or applying a pressure (positive or negative) sequence to one or more lymph nodes in the subject's neck. Lymphatic system anatomy is well know in the art. For example, the lymphatic system of the head and neck includes both lymphatic vessels and lymph nodes. Lymphatic vessels include superficial vessels that drain lymph from the scalp, face, and neck and deep vessels that are connected to deep lymph nodes. Some lymphatic vessels contain intraluminal leaflet valves that bias lymph flow toward the subject's heart. Additionally, similar to lymphatic vessels, lymph nodes are divided into two groups. The first group includes superficial lymph nodes, which receive lymph from the scalp, face, and neck. Superficial lymph nodes include, but are not limited to, occipital, mastoid, pre-auricular, parotid, submental, submandibular, and superficial cervical nodes. The second group includes deep lymph nodes, which receive the lymph from the head and neck. Deep lymph nodes include, but are not limited to, prelaryngeal, pretracheal, paratracheal, retropharyngeal, infrahyoid, jugulodigastric (tonsillar), jugulo-omohyoid and supraclavicular nodes. The lymphatic system of the head and neck also includes Waldeyer's Ring, which is a circumpharyngeal ring of mucosa-associated lymphoid tissue that surrounds the openings into the digestive and respiratory tracts. Waldeyer's Ring is made up the lingual tonsil (anteroinferiorly), the palatine and tubal tonsils (laterally), the nasopharyngeal tonsil (posterosuperiorly), as well as smaller collections of lymphoid tissue in the inter-tonsillar intervals. FIG. 15A is an illustration of the lymphatic system of the head and neck from Encyclopedia Britannica, Encyclopaedia Britannica, 2008. As shown in FIG. 15A, the lymphatic system of the head and neck includes mastoid nodes 1500A, occipital nodes 1500B, external jugular node 1500C, sternocleidomastoid nodes 1500D, jugulodigastric node 1500E, subparotid node 1500F, lateral group of deep cervical (spinal accessory) nodes 1500G, intercalated node 15001, internal jugular node 1500J, inferior deep cervical (scalene) node 1500K, thoracic duct 1500M, subclavian node 1500N, superficial parotid nodes (deep parotid nodes deep to parotid gland) 15000, facial nodes (buccal nodes) 1500P, mandibular and submandibular nodes 1500Q, submental nodes 1500R, suprahyoid node 1500S, superior thyroid nodes 1500T, anterior deep cervical nodes 1500V, juguloomohyoid nodes 1500W, anterior jugular nodes 1500X, and supraclavicular node 1500Y.

Additionally, lymph nodes of the neck have been divided into anatomic lymph node levels: Level I, submental and submandibular; Level Il, upper jugular group; Level III, middle jugular group; Level IV, lower jugular group, Level V, posterior triangle located between the sternocleidomastoid and trapezius muscle, Level VI, anterior compartment located midline between carotid sheets from the hyoid bone superiorly to the suprasternal notch inferiorly; Level VII, mediastinal lymph nodes. FIG. 15B is an illustration of the anatomic lymph node levels from Dijk et al. (2020). Surgical Complications and Referral Patterns in 567 Patients with Differentiated Thyroid Cancer in the Northern Region of the Netherlands: A Population-Based Study Towards Clinical Management Implementation. Annals of Surgical Oncology. 27. 10.1245/s10434-020-08470-1.

### Neuromuscular Electrical Stimulation

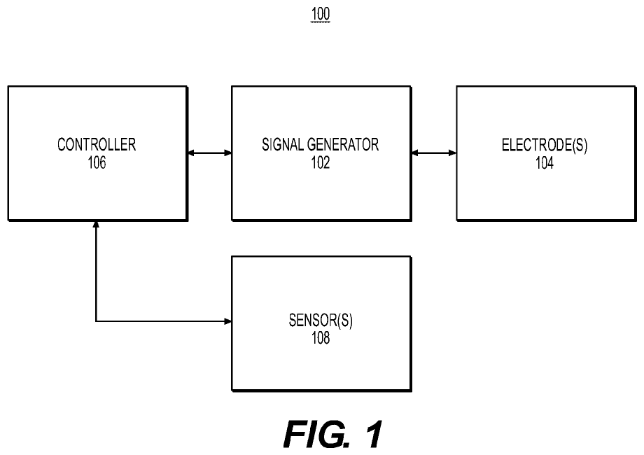

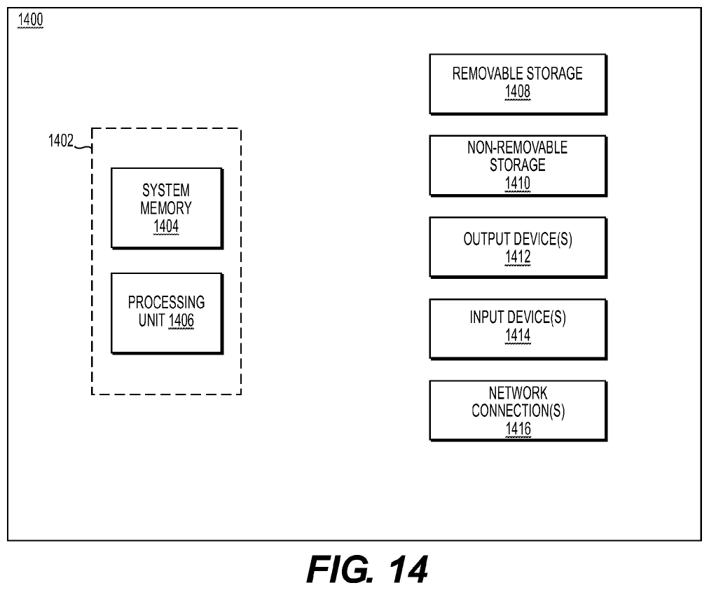

In one implementation, neuromuscular electrical stimulation (NMES) is used to improve CSF drainage. NMES is a non-invasive technique for improving CSF drainage. The head and neck lymphatic system is described above and shown in FIGS. 15A and 15B. A block diagram of a system 100 for draining CSF though a subject's neck lymphatic system is shown in FIG. 1. The system 100 includes a signal generator 102, a plurality of surface electrodes 104, and a controller 106. The signal generator 102 is configured to generate NMES and is operably connected to the surface electrodes 104. The surface electrodes 104 are configured to deliver transcutaneous NMES to a subject. Skin surface electrodes are known in the art and therefore not described in further detail herein. Example skin surface electrodes are LONG LIFE PADS electrodes manufactured by Omron Corporation of Osaka, Japan. It should be understood that LONG LIFE PADS electrodes are provided only as an example and that other skin surface electrodes can be used with the systems and methods described herein. The signal generator 102 can be a voltage source or current source. Optionally, the signal generator 102 may include programmable logic, e.g., a processor and memory such as the most basic configuration of example computing device 1400 shown in FIG. 14. The programmable logic can be programmed to control operation of the signal generator 102. Signal generators are known in the art and therefore not described in further detail herein. An example signal generator is the COMPEX SPORT ELITE 3.0 muscle stimulator manufactured by DJO Global Switzerland Sàrl of Ecublens, Switzerland. It should be understood that the COMPEX SPORT ELITE 3.0 muscle stimulator is provided only as an example and that other signal generators can be used with the systems and methods described herein. Implantable signal generators are also known in the art, e.g., the networked neuroprosthetic system (NNP) from the Cleveland FES Center. The signal generator 102 and surface electrodes 104 can be coupled using any wired or wireless (e.g., inductive, radiofrequency (RF)) link. In some implementations, the signal generator 102 is implanted in a subject's body. In other implementations, the signal generator 102 is external to the subject's body. The controller 106 is operably connected to the signal generator 102, for example, using any wired, wireless, or optical link that facilitates exchange of data between the controller 106 and the signal generator 102. The controller 106 can optionally be example computing device 1400 shown in FIG. 14.

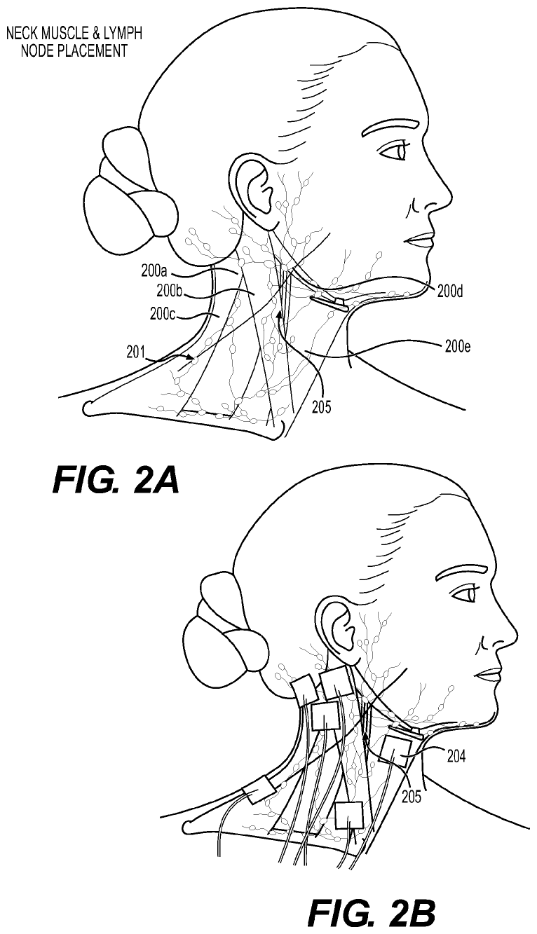

The controller 106 is configured to control the signal generator 102 to deliver, via the skin surface electrodes 104, a NMES signal to at least one muscle in the subject's neck. For example, the signal generator 102 generates the NMES signal, and the controller 106 transmits control signals to the signal generator 102, where the control signals determine the characteristics (e.g., waveform shape, magnitude of current/voltage, frequency, duration, etc.) of the NMES signal. As described herein, the NMES signal can be delivered to one or more of musculus platysma, sternocleidomastoid, or trapezius. In some implementations, the NMES signal is delivered to a single muscle in the subject's neck. In some implementations, the NMES signals are delivered to multiple muscles in the subject's neck. Multiple NMES signals can be delivered to different muscles simultaneously or at different times. Additionally, the surface electrodes 104 can be arranged on the patient's neck, for example, as shown in FIGS. 2A and 2B. FIG. 2A illustrates neck muscle 200 and lymph node 201 arrangement in a subject. The neck muscles shown in FIG. 2A include splenius capitis muscle 200a, sternocleidomastoid muscle 200b, trapezius muscle 200c, digastric muscle 200d, and platysma muscle 200e. Neck muscles are collectively referred to herein as neck muscle or muscles 200. It should be understood that only a single lymph node is labeled with a reference number in FIG. 2A for clarity. Placement of the surface electrodes 204 relative to the subject's neck muscles and lymph nodes are shown in FIG. 2B. It should be understood that only a single surface electrode 204 is labeled with a reference number in FIG. 2B for clarity. The subject's carotid bifurcation 205 is also labeled in FIGS. 2A and 2B. The carotid artery bifurcation is the point where the carotid artery divides into internal and external branches. It is typically located at about the level of the third or fourth cervical vertebra (C3 or C4). It should be understood that FIG. 2B is provided only as an example and that the surface electrodes can be arranged differently than shown in FIG. 2B. Optionally, the surface electrodes are placed to target deep lymph nodes and/or the myodural bridge.

Referring again to FIG. 1, the NMES signal is configured to induce a plurality of muscle contractions. Such muscle contractions are configured to squeeze at least one lymph node to create a pumping force, which is configured to direct CSF flow in a proximal direction. As used herein, the proximal direction is the direction away from the subject's head and toward the subject's heart. The pumping force constricts valve endowed lymphatic vessels, which provides CSF flow directionality away from the subject's head and toward the heart. It should be understood that lymphatic vessels contain intraluminal leaflet valves that bias flow toward the subject's heart. This disclosure contemplates that the NMES signal is delivered over a time period that improves CSF drainage. For example, the NMES signal can optionally be delivered for a period of about 5-60 minutes in some implementations. It should be understood that the NMES signal can be delivered for less than 5 minutes or greater than 60 minutes in other implementations.

As described herein, the NMES signal includes at least one burst of pulses. The burst of pulses can optionally include rectangular pulses. The rectangular pulses can be monophasic or biphasic pulses. Additionally, each of the rectangular pulses can have a duration of about 0.5-1,000 microseconds (usec). For example, each of the rectangular pulses optionally has a duration of about 500 usec. It should be understood that the durations of the rectangular pulses provided above are only examples and that the pulse duration may have other values. Alternatively or additionally, the burst of pulses can include sinusoidal pulses. The sinusoidal pulses can have a frequency of about 5-150 Hertz (Hz). For example, the sinusoidal pulses optionally have a frequency of about 10 Hz. It should be understood that the frequencies of the sinusoidal pulses provided above are only examples and that the frequency may have other values. Alternatively or additionally, in some implementations, the NMES signal is delivered with a current of about 10-100 milliamps (mA). In some implementations, the NMES signal includes a single burst of pulses. In other implementations, the NMES signal includes a plurality of bursts of pulses. Optionally, the plurality of bursts of pulses is a series of about 5-50 bursts of pulses. It should be understood that 5-50 bursts of pulses is provided only as an example and that the series may include less than 5 bursts of pulses or more than 50 bursts of pulses. The series can be delivered with a time delay (e.g., optionally 5-20 seconds delay) between bursts of pulses. Alternatively or additionally, the series is optionally delivered at a frequency of about 0.02-1 Hz.

Optionally, in some implementations, the system 100 further includes a sensor 108 configured to detect muscular contractions. The sensor 108 is operably connected to the controller 106, for example, using any wired, wireless, or optical link that facilitates exchange of data between the controller 106 and the sensors 108. For example, the sensor 108 is optionally a strain gauge. A strain gauge can convert a muscular contraction into an electrical signal (e.g., change in resistance). It should be understood that a strain gauge is provided only as an example. This disclosure contemplates that the sensor 108 can be another type of sensor configured to detect muscular contractions. Additionally, the system 100 can optionally include more than one, i.e., a plurality of sensors 108 in some implementations. For example, this disclosure contemplates that one or more sensors 108 can be placed in proximity to each of the one or more muscles being stimulated as described herein. The controller 106 can be further configured to receive a feedback signal from the sensor 108. The feedback signal includes information related to a contraction state of the at least one muscle (i.e., the muscle to which the NMES stimulation is delivered). The controller 106 can be further configured to increase a magnitude of the NMES signal current until detecting contraction of the at least one muscle. This can be accomplished by transmitting an appropriate control signal from the controller 106 to the signal generator 102. For example, the magnitude of the NMES signal current is optionally increased in a step-wise basis from about 2.5 mA until muscle contraction is induced. This current level is understood to be the minimum current that induces muscle contraction. It should be understood that the initial current (i.e., 2.5 mA) and step-wise increase can have any value. The controller 106 can be further configured to increase (and optionally in a step-wise manner) the magnitude of the NMES signal current above the minimum current that induces contraction of the at least one muscle. This can be accomplished by transmitting an appropriate control signal from the controller 106 to the signal generator 102. For example, the magnitude of the NMES signal current can optionally be increased about 10-50% above the minimum current. It should be understood that 10-50% above the minimum current is provided only as an example and that the NMES signal current may have other values, including less than 10% above the minimum current when the subject cannot tolerate a current of greater magnitude.

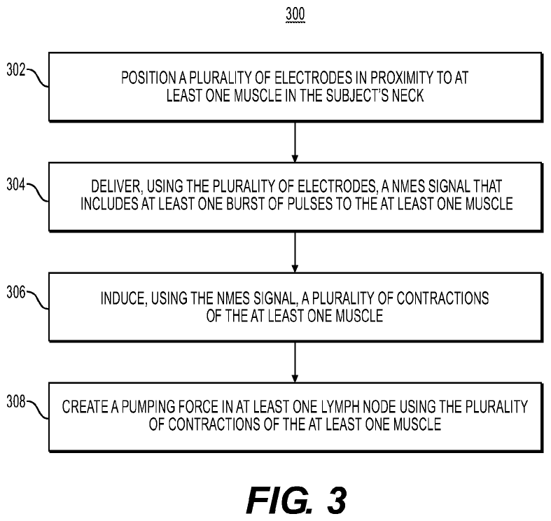

Referring now to FIG. 3, example operations 300 for draining CSF though a subject's neck lymphatic system are shown. This disclosure contemplates using the system 100 of FIG. 1 to perform the operations of FIG. 3. It should be understood that the operations of FIG. 3 can be performed using a different system than shown in FIG. 1. At step 302, a plurality of surface electrodes (e.g., surface electrodes 104 of FIG. 1; surface electrodes 204 of FIG. 2B) are positioned in proximity to at least one muscle in the subject's neck. As described above, the muscle can optionally be one or more of musculus platysma, sternocleidomastoid, or trapezius. Example neck muscles, lymph nodes, and surface electrode placement are also shown in FIGS. 2A and 2B. At step 304, a NMES signal is delivered, using the surface electrodes, to the at least one muscle. As described above, the NMES signal is generated using a signal generator (e.g., signal generator 102 of FIG. 1). Characteristics of the NMES signal are described above and therefore not described in detail below. Additionally, in some implementations, the NMES signal is optionally controlled in response to a feedback signal detected by a sensor (e.g., sensor 108 of FIG. 1) as described herein. At step 306, a plurality of contractions of the at least one muscle are induced by the NMES signal. At step 308, a pumping force is created in at least one lymph node due to the plurality of contractions of the at least one muscle. The pumping force directs CSF flow through the subject's lymphatic system (e.g., one or more lymph nodes and vessels in the neck) in a proximal direction. For example, as described above, muscle contractions squeeze the subject's lymph node(s) to create a pumping force, which is configured to direct CSF flow away from the subject's head and toward the heart (i.e., the proximal direction as described herein).

In some implementations, the operations 300 are used to treat a disease or condition in the subject. For example, the disease or condition is treated by directing CSF flow in the proximal direction. The disease or condition can included, but is not limited to, hydrocephalus, a neurodegenerative disorder, or a sleep disorder. Alternatively or additionally, in some implementations, the operations 300 are used to improve the subject's sleep health, for example, by directing CSF flow in the proximal direction. Alternatively or additionally, in some implementations, the operations 300 are used to improve circulation of neurotrophic agents, for example, by directing CSF flow in the proximal direction. Alternatively or additionally, in some implementations, the operations 300 are used to improve removal of biological waste agents, for example, by directing CSF flow in the proximal direction.

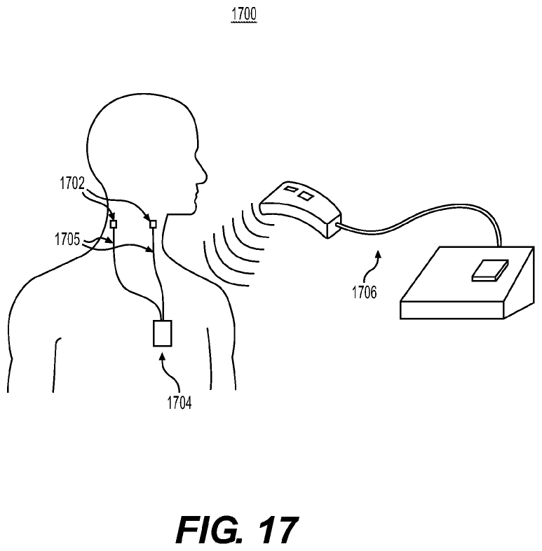

In the implementations described above with regard to FIGS. 1 and 3 above, NMES is delivered via surface electrodes (e.g., transcutaneous NMES is delivered). This disclosure also contemplates delivering NMES using an implantable system to improve CSF drainage, for example, using an implantable signal generator and implantable electrodes. Referring now to FIG. 17, a diagram of a system 1700 for draining CSF though a subject's neck lymphatic system is shown. The system 1700 includes a plurality of implantable electrodes 1702, an implantable signal generator 1704, and a remote controller 1706. The implantable signal generator 1704 is configured to generate NMES and is operably connected to the implantable electrodes 1702. The implantable electrodes 1702 are configured to deliver subcutaneous NMES to a subject. The implantable electrodes 1702 can be arranged on the patient's neck, for example, in positions relative to the subject's neck muscles and lymph nodes as described above.

The implantable signal generator 1704 may include programmable logic, e.g., a processor and memory such as the most basic configuration of example computing device 1400 shown in FIG. 14. The programmable logic can be programmed to control operation of the implantable signal generator 1704. The implantable signal generator 1704 and implantable electrodes 1702 can be coupled using any wired or wireless (e.g., inductive, radiofrequency (RF)) link 1705. The remote controller 1706 is operably connected to the implantable signal generator 1704, for example, using any wireless or optical link that facilitates exchange of data between the remote controller 1706 and the implantable signal generator 1704. The remote controller 1706 can optionally be example computing device 1400 shown in FIG. 14. Implantable signal generators and electrodes are known in the art. For example, the BarostimNEO system (which is for cardiac stimulation) of CVRx Inc. of Minneapolis, MN is an example system including implantable signal generator and electrodes. It should be understood that the BarostimNEO system is provided only as an example implantable and that other systems may be used to provide subcutaneous NMES.

The remote controller 1706 is configured to control the implantable signal generator 1704 to deliver, via the implantable electrodes 1702, a NMES signal to at least one muscle in the subject's neck. For example, the remote controller 1706 can be configured to modify the NMES signal (e.g., pulse forms, frequency, duty cycle, waveform shape, magnitude of current/voltage, frequency, duration, etc.). The remote controller 1706 can transmits control signals to the implantable signal generator 1704. Additionally, the implantable signal generator 1704 generates the NMES signal according to such control signals received from the remote controller 1706. Similarly as described above, the subcutaneous NMES signal is configured to induce a plurality of muscle contractions. Such muscle contractions are configured to squeeze at least one lymph node to create a pumping force, which is configured to direct CSF flow in a proximal direction. As used herein, the proximal direction is the direction away from the subject's head and toward the subject's heart. The pumping force constricts valve endowed lymphatic vessels, which provides CSF flow directionality away from the subject's head and toward the heart. This disclosure contemplates that the subcutaneous NMES stimulation may have one or more of the characteristics (e.g., pulse shape, duration, frequency, current, etc.) as described above.

Example operations for draining CSF though a subject's neck lymphatic system using subcutaneous NMES are now described. This disclosure contemplates using the system 1700 of FIG. 17 to perform these operations. It should be understood that these operations can be performed using a different system than shown in FIG. 17. A plurality of implantable electrodes (e.g., implantable electrodes 1702 of FIG. 17) are positioned in proximity to at least one muscle in the subject's neck. As described above, the muscle can optionally be one or more of musculus platysma, sternocleidomastoid or trapezius. Additionally, a subcutaneous NMES signal is delivered, using the implantable electrodes, to the at least one muscle. As described above, the subcutaneous NMES signal is generated using a signal generator (e.g., implantable signal generator 1704 of FIG. 17). Additionally, in some implementations, the subcutaneous NMES signal is optionally controlled in response to a feedback signal detected by a sensor as described herein. Additionally, a plurality of contractions of the at least one muscle are induced by the NMES signal, and a pumping force is created in at least one lymph node due to the plurality of contractions of the at least one muscle. The pumping force directs CSF flow through the subject's lymphatic system (e.g., one or more lymph nodes and vessels in the neck) in a proximal direction. For example, as described above, muscle contractions squeeze the subject's lymph node(s) to create a pumping force, which is configured to direct CSF flow away from the subject's head and toward the heart (i.e., the proximal direction as described herein).

### Mechanical Actuation

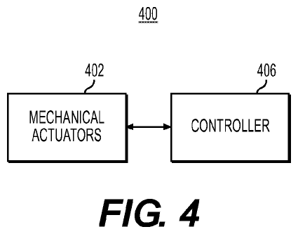

In another implementation, mechanical actuators are used to improve CSF drainage. Mechanical actuation is a non-invasive technique for improving CSF drainage. The head and neck lymphatic system is described above and shown in FIGS. 15A and 15B. A block diagram of a system 400 for draining CSF though a subject's neck lymphatic system is shown in FIG. 4. The system includes a plurality of mechanical actuators 402 and a controller 406 operably connected to the plurality of mechanical actuators 402. The controller 406 is operably connected to the mechanical actuators 402, for example, using any wired, wireless, or optical link that facilitates exchange of data between the controller 406 and the mechanical actuators 402. The controller 406 can optionally be example computing device 1400 shown in FIG. 14.

The controller 406 is configured to sequentially activate the mechanical actuators 402 to exert a positive pressure sequence in proximity to at least one lymph node in the subject's neck, which is located proximally with respect to the subject's carotid artery bifurcation. In some implementations, the mechanical actuators 402 are inflatable and activated by energizing a pump or other mechanism configured to pressurize/depressurize the mechanical actuators with an air or a fluid as described below. Example pneumatic compression systems are the FLEXITOUCH PLUS and ENTRE systems manufactured by Tactile Medical of Minneapolis, MN. It should be understood that the FLEXITOUCH PLUS and ENTRE systems are provided only as examples and that other pumps or mechanisms can be used with the systems and methods described herein. In other implementations, the mechanical actuators 402 are motorized or piezoelectric and activated by an electrical signal. It should be understood that the controller 406 can be configured to provide appropriate control signals to actuate the mechanical actuators 402. In some implementations, the mechanical actuators 402 are activated sequentially in the proximal direction. Optionally, each of the mechanical actuators 402 is activated individually such that the mechanical actuators 402 are actuated sequentially. As used herein, the proximal direction is the direction away from the subject's head and toward the subject's heart. The at least one lymph node where the positive pressure sequence is applied is located below the carotid artery bifurcation, i.e., in the direction towards the subject's thoracic spine. The positive pressure sequence is configured to squeeze the at least one lymph node to create a pumping force, and the pumping force is configured to direct CSF flow away from the subject's head and toward the subject's heart (i.e., the proximal direction). As described herein, the pumping force constricts lymphatic vessels, which contain intraluminal leaflet valves that bias flow toward the subject's heart, causing CSF to flow away from the subject's head and toward the heart. In some implementations, the mechanical actuators 402 are activated repeatedly to exert a series of positive pressure sequences.

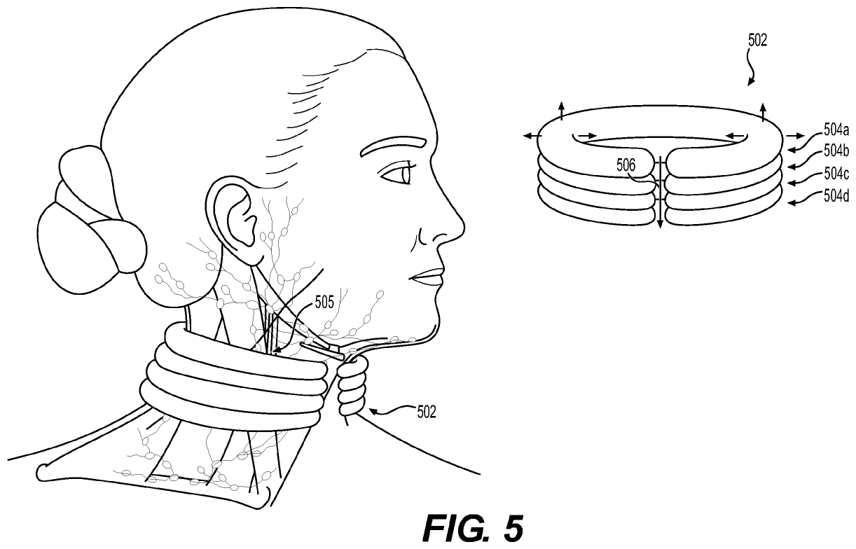

Each of the mechanical actuators 402 optionally includes an inflatable member. In this implementation, the mechanical actuators 402 also include a pump or other mechanism for inflating and deflating the inflatable members. This disclosure contemplates that inflatable members can be filled with air or other fluid (e.g., another gas or liquid). The inflatable members and pump or other mechanism are arranged in fluid connection, e.g., such that fluid can move therebetween. An example collar 502 that includes a plurality of inflatable members 504a-504d (referred to herein collectively as “inflatable members 504”) is shown in FIG. 5. It should be understood that the number of inflatable members 504 shown in FIG. 5 is provided only as an example. This disclosure contemplates that the collar 502 can include more or less than four inflatable members 504. Additionally, in some implementations, the collar 502 shown in FIG. 5 is optionally arranged entirely below the subject's carotid artery bifurcation 505. This ensures that mechanical actuation of the collar 502 does not have an adverse impact on blood circulation by exercising pressure on the carotid sinus, where arterial pressure sensors are located. As described above, the carotid artery bifurcation is the point where the carotid artery divides into internal and external branches, and it is usually located near vertebra C3 or C4 of the subject. Thus, the collar 502 can be used to exert a positive pressure sequence in proximity to at least one lymph node that is located proximally with respect to the subject's carotid artery bifurcation. Alternatively, in some implementations, the collar 502 optionally includes a noninflatable segment that can be positioned over the carotid artery bifurcation 505 to avoid external pressure on the carotid sinus, while the inflatable members 504 are activated. In this implementation, the collar 502 (or portions thereof) may be arranged at or above the subject's carotid artery bifurcation 505 without exercising pressure on the carotid sinus. Optionally, the inflatable members 504a-504d are activated sequentially in the proximal direction, e.g., from top to bottom. For example, inflatable member 504a is activated first followed by simultaneous deflation of inflatable member 504a and inflation of inflatable member 504b. Thereafter, inflatable member 504b is deflated simultaneously with inflation of inflatable member 504c. Thereafter, inflatable member 504c is deflated simultaneously with inflation of inflatable member 504d. This creates a positive pressure sequence, e.g., a pressure wave. The positive pressure sequence squeezes the at least one lymph node to create a pumping force that direct CSF flow in the proximal direction, i.e., away from the subject's head and toward the subject's heart. Arrow 506 in FIG. 5 shows the proximal direction.

Optionally, an internal pressure of the inflatable members 504 is regulated between about 2 millimeters of mercury (mmHg) and 10 mmHg during a mechanical activation cycle. For example, the internal pressure of the inflatable members 504 can optionally be regulated to about 5 mmHg during the mechanical activation cycle. It should be understood that an internal pressure of 2-10 mmHg is provided only as an example and that the internal pressure may be regulated to less than 2 mmHg or more than 10 mmHg. A duration of the mechanical activation cycle is optionally about 500 milliseconds (msec)-5 seconds. It should be understood that a duration of 500 msec-5 seconds is provided only as an example and that the duration may be less than 500 msec or more than 5 seconds. Additionally, this disclosure contemplates that mechanical actuation can be applied over a time period that improves CSF drainage. For example, a series of positive pressure sequences can be applied. Positive pressure sequences can optionally be applied repeatedly as described above over a period of time (e.g., 5-15 minutes). This process can optionally be repeated periodically (e.g., every 30 minutes) as needed to improve CSF drainage.

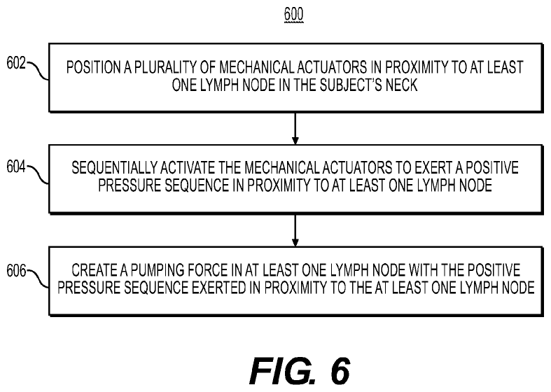

Referring now to FIG. 6, example operations 600 for draining CSF though a subject's neck lymphatic system are shown. This disclosure contemplates using the system 400 of FIG. 4 to perform the operations of FIG. 6. It should be understood that the operations of FIG. 6 can be performed using a different system than shown in FIG. 4. At step 602, a plurality of mechanical actuators (e.g., mechanical actuators 402 of FIG. 4; inflatable members 504 of FIG. 5) in proximity to at least lymph node in the subject's neck. As described above, the at least one lymph node is located proximally with respect to the subject's carotid artery bifurcation. Example mechanical actuator placement is also shown in FIG. 5. At step 604, the mechanical actuators are sequentially activated to exert a positive pressure sequence (e.g., a pressure wave) in proximity to the at least one lymph node. Mechanical actuator activation parameters are described above and therefore not described in further detail below. At step 606, a pumping force is created in at least one lymph node due to the positive pressure sequence that is exerted in proximity to the at least one lymph node. The pumping force directs CSF flow through the subject's lymphatic system (e.g., one or more lymph nodes and vessels in the neck) in a proximal direction. For example, as described above, mechanical actuation squeezes the subject's lymph node(s) to create a pumping force, which is configured to direct CSF flow away from the subject's head and toward the heart (i.e., the proximal direction as described herein).

As described above, in some implementations, the operations 600 are used to treat a disease or condition in the subject. For example, the disease or condition is treated by directing CSF flow in the proximal direction. The disease or condition can included, but is not limited to, hydrocephalus, a neurodegenerative disorder, or a sleep disorder. Alternatively or additionally, in some implementations, the operations 600 are used to improve the subject's sleep health, for example, by directing CSF flow in the proximal direction. Alternatively or additionally, in some implementations, the operations 600 are used to improve circulation of neurotrophic agents, for example, by directing CSF flow in the proximal direction. Alternatively or additionally, in some implementations, the operations 600 are used to improve removal of biological waste agents, for example, by directing CSF flow in the proximal direction.

### Vacuum Suction

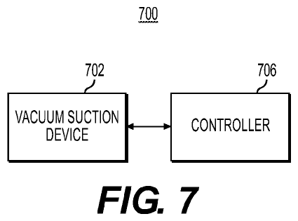

In another implementation, a vacuum suction device is used to improve CSF drainage. Vacuum suction is a non-invasive technique for improving CSF drainage. The head and neck lymphatic system is described above and shown in FIGS. 15A and 15B. A block diagram of a system 700 for draining CSF though a subject's neck lymphatic system is shown in FIG. 7. The system includes a vacuum suction device 702 and a controller 706 operably connected to the vacuum suction device 702. The controller 706 is operably connected to the vacuum suction device 702, for example, using any wired, wireless, or optical link that facilitates exchange of data between the controller 706 and the vacuum suction device 702. The controller 706 can optionally be example computing device 1400 shown in FIG. 14.

The controller 706 is configured to sequentially activate the vacuum suction device 702 to exert a negative pressure sequence in proximity to at least one lymph node in the subject's neck, which is located proximally with respect to the subject's carotid artery bifurcation. As described below, the vacuum suction device 702 can include an inflatable member and can be activated by energizing a pump or other mechanism configured to pressurize/depressurize the inflatable member with an air or fluid. It should be understood that the controller 706 can be configured to provide appropriate control signals to actuate the vacuum suction device 702. As used herein, the proximal direction is the direction away from the subject's head and toward the subject's heart. The at least one lymph node where the negative pressure sequence is applied is located below the carotid artery bifurcation, i.e., in the direction towards the subject's thoracic spine. The negative pressure sequence is configured to expand the at least one lymph node, which creates a suction that draws fluid into the at the at least one lymph node. The suction is therefore configured to direct CSF flow away from the subject's head and toward the subject's heart (i.e., the proximal direction). In some implementations, the vacuum suction device 702 is activated repeatedly to exert a series of negative pressure sequences.

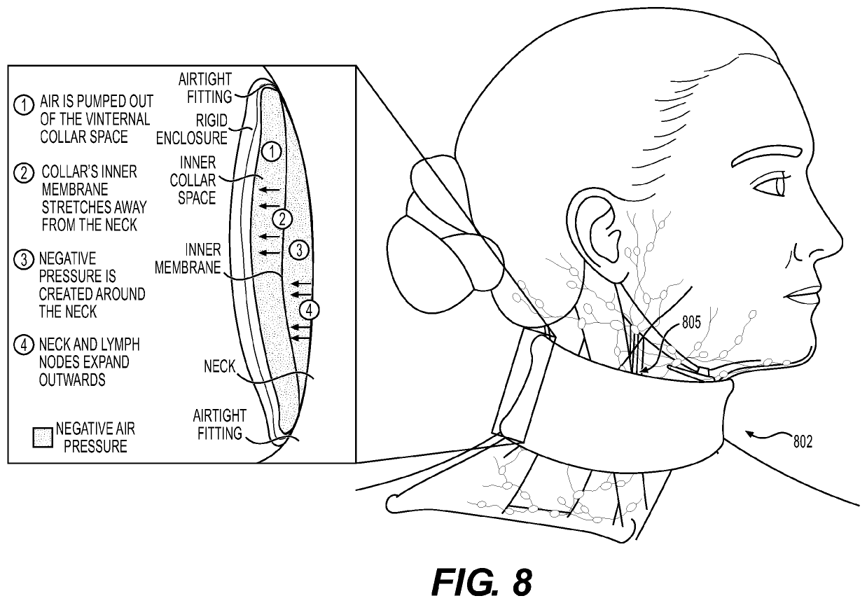

The vacuum suction device 702 includes an inflatable member and a pump or other mechanism for inflating and deflating the inflatable member. This disclosure contemplates that inflatable member can be pressurized/depressurized using air or other fluid (e.g., another gas or liquid). The inflatable member and pump or other mechanism are arranged in fluid connection, e.g., such that fluid can move therebetween. An example negative pressure collar 802 that includes an inflatable member is shown in FIG. 8. It should be understood that the number of inflatable members shown in FIG. 8 is provided only as an example. This disclosure contemplates that the collar 802 can include more than one inflatable member. Additionally, the collar 802 shown in FIG. 8 is arranged entirely below the subject's carotid artery bifurcation 805. This ensures that actuation of the negative pressure collar 802 does not have an adverse impact on blood circulation by exercising pressure on the carotid sinus, where arterial pressure sensors are located. As described above, the carotid artery bifurcation is the point where the carotid artery divides into internal and external branches, and it is usually located near vertebra C3 or C4 of the subject. Thus, the negative pressure collar 802 can be used to create a suction in proximity to at least one lymph node that is located proximally with respect to the subject's carotid artery bifurcation. Alternatively, in some implementations, the collar 802 optionally includes a noninflatable segment that can be positioned over the carotid artery bifurcation 805 to avoid vacuum suction on the carotid sinus, while the collar 802 is activated. In this implementation, the collar 802 (or portions thereof) may be arranged at or above the subject's carotid artery bifurcation 805 without applying vacuum on the carotid sinus.

Optionally, a vacuum of the vacuum suction device 702 is regulated between about 3 mmHg and 5 mmHg during a vacuum activation cycle. It should be understood that a vacuum of 3-5 mmHg is provided only as an example and that the vacuum may be regulated to less than 5 mmHg or more than 3 mmHg. A duration of the vacuum activation cycle is about 500 msec-2 seconds. It should be understood that a duration of 500 msec-2 seconds is provided only as an example and that the duration may be less than 500 msec or more than 2 seconds. Additionally, this disclosure contemplates that vacuum suction can be applied over a time period that improves CSF drainage. For example, negative pressure sequences can optionally be applied repeatedly as described above over a period of time (e.g., every 1-10 minutes) as needed to improve CSF drainage.

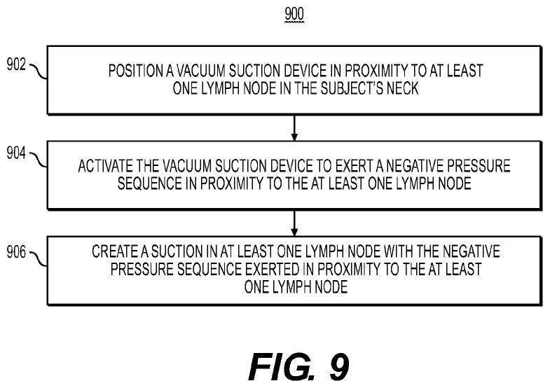

Referring now to FIG. 9, example operations 900 for draining CSF though a subject's neck lymphatic system are shown. This disclosure contemplates using the system 700 of FIG. 7 to perform the operations of FIG. 9. It should be understood that the operations of FIG. 9 can be performed using a different system than shown in FIG. 7. At step 902, a vacuum suction device (e.g., vacuum suction device 702 of FIG. 7; negative pressure collar 802 of FIG. 8) in proximity to at least lymph node in the subject's neck. As described above, the at least one lymph node is located proximally with respect to the subject's carotid artery bifurcation. Example vacuum suction device placement is also shown in FIG. 8. At step 904, the vacuum suction device is activated to exert a negative pressure sequence in proximity to the at least one lymph node. Vacuum suction device activation parameters are described above and therefore not described in further detail below. As described herein, the negative pressure sequence expands the at least one lymph node. As a result, at step 906, a suction is created in at least one lymph node. The suction directs CSF flow through the subject's lymphatic system (e.g., one or more lymph nodes and vessels in the neck) in a proximal direction, i.e., away from the subject's head and toward the heart).

As described above, in some implementations, the operations 900 are used to treat a disease or condition in the subject. For example, the disease or condition is treated by directing CSF flow in the proximal direction. The disease or condition can included, but is not limited to, hydrocephalus, a neurodegenerative disorders, or a sleep disorder. Alternatively or additionally, in some implementations, the operations 900 are used to improve the subject's sleep health, for example, by directing CSF flow in the proximal direction. Alternatively or additionally, in some implementations, the operations 900 are used to improve circulation of neurotrophic agents, for example, by directing CSF flow in the proximal direction. Alternatively or additionally, in some implementations, the operations 900 are used to improve removal of biological waste agents, for example, by directing CSF flow in the proximal direction.

### Implantable Device

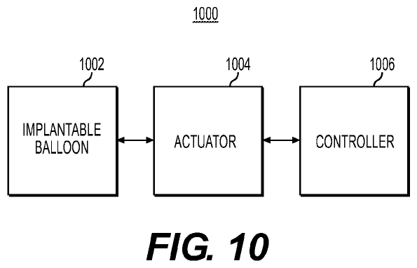

In another implementation, an implantable balloon is used to improve CSF drainage. A block diagram of a system 1000 for draining CSF though a subject's neck lymphatic system is shown in FIG. 10. The system includes an implantable balloon 1002, an actuator 1004, and a controller 1006 operably connected to the actuator 1004. The implantable balloon 1002 and the actuator 1004 are arranged in fluid connection, e.g., such that fluid can move therebetween. In some implementations, the actuator 1004 is implanted in a subject's body. In other implementations, the actuator 1004 is external to the subject's body. The controller 1006 is operably connected to the actuator 1004, for example, using any wired, wireless, or optical link that facilitates exchange of data between the controller 1006 and the actuator 1004. For example, the actuator 1004 can be energized by the controller 1006 to inflate/deflate the inflatable balloon 1002 using air or other fluid (e.g., another gas or liquid). For example, in some implementations, the actuator 1004 can include a small volume pump in fluid connection with a plurality of valve-equipped cylinders. A first cylinder is in fluid connection with the pump, a second cylinder is in fluid connection with the implantable balloon 1002, and the first and second cylinders are connected via a regulatory valve. Beating-like pressure changes are created by directing fluid through the first and second cylinders and into the implantable balloon 1002. It should be understood that the controller 1006 can be configured to provide appropriate control signals to inflate/deflate the inflatable balloon 1002, for example, by energizing the pump and/or valves. This disclosure contemplates that the inflatable balloon 1002 can be made from a biologically inert material such as polypropylene, polyvinyl, silicone or thermoplastics. It should be understood that such materials become softer at body temperature and also return to the initial shape after deflation. The controller 1006 can optionally be example computing device 1400 shown in FIG. 14.

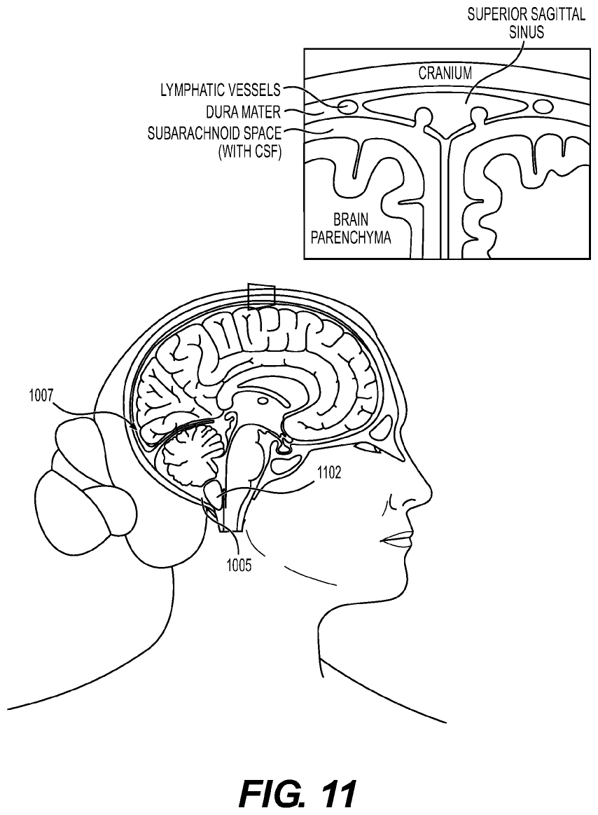

The controller 1006 is configured energize the actuator 1004 and to inflate the implantable balloon 1002 to compress the subject's cisterna magna. It should be understood that the controller 1006 can be configured to provide appropriate control signals to the actuator 1004 to inflate/deflate the inflatable balloon 1002. As shown in FIG. 11, an implantable balloon 1102 is located at a junction of the subject's cisterna magna 1005 and perispinal subarachnoid space 1007. The cisterna magna 1005 (or cerebellomedullary cistern) is the largest subarachnoid cistern. Inflation of the implantable balloon 1102 pushes the CSF along the perispinal subarachnoid space 1007 and further along the nerve roots and into the subject's neck lymphatic system. In other words, compression of the cisterna magna 1005 causes CSF to flow toward the subject's perispinal subarachnoid space 1007.

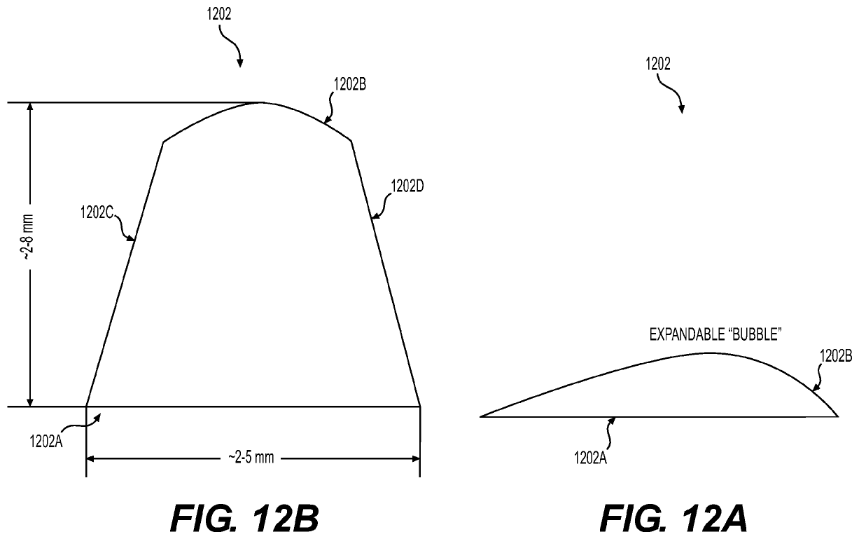

In some implementations, the implantable balloon has a trapezoidal shape. For example, FIGS. 12A and 12B illustrate an implantable balloon 1202 having a trapezoidal shape in both the deflated state (FIG. 12A) and the inflated state (FIG. 12B). As shown in FIG. 12B, the implantable balloon 1202 has a pair of parallel bases 1202a, 1202b and a pair of legs 1202c, 1202d extending between the pair of parallel bases 1202a, 1202b. Optionally, a first base 1202a, which has the longer length, is thicker than a second base 1202b, which has the shorter length. This is shown by differing line thicknesses of bases 1202a, 1202b in FIG. 12A. Different base thicknesses ensures that the first base 1202a does not inflate and instead exerts pressure on the underlying dura matter. The first base 1202a is implanted toward the subject's cerebellum (see FIG. 11). Additionally, this disclosure contemplates that the size of the implantable balloon 1202 can be determined based on medical imaging (e.g., MRI or CT imaging) of the subject, for example, to determine the space between cerebellar vermis and edge of the foramen magna. FIG. 12B illustrates example sizes for the implantable balloon 1202, e.g., width of about 2-5 millimeters (mm) and expandable height of about 2-8 mm. It should be understood that the implantable balloon 1202 may have a size other than shown in FIG. 12B.

Referring again to FIG. 10, the controller 1006 can be further configured to control inflation parameters for the implantable balloon 1002. For example, in some implementations, an internal pressure of the implantable balloon 1002 is regulated from about 0 mmHg to about 10-15 mmHg during inflation. Optionally, a rate of inflation is about 1 mmHg per second. It should be understood that the internal pressure and rate of inflation above are provided only as examples and may have other values.

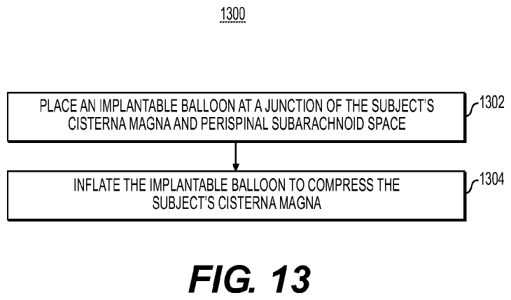

Referring now to FIG. 13, example operations 1300 for draining CSF though a subject's neck lymphatic system are shown. This disclosure contemplates using the system 1000 of FIG. 10 to perform the operations of FIG. 13. It should be understood that the operations of FIG. 13 can be performed using a different system than shown in FIG. 10. At step 1302, an implantable balloon (e.g., implantable balloon 1002 of FIG. 10; implantable balloon 1102 of FIG. 11; implantable balloon 1202 of FIGS. 12A-12B) is placed at a junction of the subject's cisterna magna and perispinal subarachnoid space. This disclosure contemplates that the implantable balloon can be placed using surgical techniques known in the art. Such surgical techniques include, but are not limited to, a midline suboccipital approach or a minimally invasive approach through tubing inserted through the foramen magnum with or without or minimal resection of the upper edge of the foramen magnum. Example inflatable balloon placement is also shown in FIG. 11. At step 1304, the implantable balloon is inflated to compress the subject's cisterna magna. Inflation parameters are described above and therefore not described in further detail below. The compression is configured to direct CSF flow toward the subject's perispinal subarachnoid space. For example, inflation of the implantable balloon pushes the CSF along the perispinal subarachnoid space and further along the nerve roots and into the subject's neck lymphatic system.

As described above, in some implementations, the operations 1300 are used to treat a disease or condition in the subject. For example, the disease or condition is treated by directing CSF flow in the proximal direction. The disease or condition can included, but is not limited to, hydrocephalus, a neurodegenerative disorder, or a sleep disorder. Alternatively or additionally, in some implementations, the operations 1300 are used to improve the subject's sleep health, for example, by directing CSF flow in the proximal direction. Alternatively or additionally, in some implementations, the operations 1300 are used to improve circulation of neurotrophic agents, for example, by directing CSF flow in the proximal direction. Alternatively or additionally, in some implementations, the operations 1300 are used to improve removal of biological waste agents, for example, by directing CSF flow in the proximal direction.

It should be appreciated that the logical operations described herein with respect to the various figures may be implemented (1) as a sequence of computer implemented acts or program modules (i.e., software) running on a computing device (e.g., the computing device described in FIG. 14), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

### Example Computing Device

Referring to FIG. 14, an example computing device 1400 upon which the methods described herein may be implemented is illustrated. It should be understood that the example computing device 1400 is only one example of a suitable computing environment upon which the methods described herein may be implemented. Optionally, the computing device 1400 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 1400 typically includes at least one processing unit 1406 and system memory 1404. Depending on the exact configuration and type of computing device, system memory 1404 may be volatile (such as random access memory (RAM)), non-volatile (such as read-only memory (ROM), flash memory, etc.), or some combination of the two. This most basic configuration is illustrated in FIG. 14 by dashed line 1402. The processing unit 1406 may be a standard programmable processor that performs arithmetic and logic operations necessary for operation of the computing device 1400. The computing device 1400 may also include a bus or other communication mechanism for communicating information among various components of the computing device 1400.

Computing device 1400 may have additional features/functionality. For example, computing device 1400 may include additional storage such as removable storage 1408 and non-removable storage 1410 including, but not limited to, magnetic or optical disks or tapes. Computing device 1400 may also contain network connection(s) 1416 that allow the device to communicate with other devices. Computing device 1400 may also have input device(s) 1414 such as a keyboard, mouse, touch screen, etc. Output device(s) 1412 such as a display, speakers, printer, etc. may also be included. The additional devices may be connected to the bus in order to facilitate communication of data among the components of the computing device 1400. All these devices are well known in the art and need not be discussed at length here.

The processing unit 1406 may be configured to execute program code encoded in tangible, computer-readable media. Tangible, computer-readable media refers to any media that is capable of providing data that causes the computing device 1400 (i.e., a machine) to operate in a particular fashion. Various computer-readable media may be utilized to provide instructions to the processing unit 1406 for execution. Example tangible, computer-readable media may include, but is not limited to, volatile media, non-volatile media, removable media and non-removable media implemented in any method or technology for storage of information such as computer readable instructions, data structures, program modules or other data. System memory 1404, removable storage 1408, and non-removable storage 1410 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1406 may execute program code stored in the system memory 1404. For example, the bus may carry data to the system memory 1404, from which the processing unit 1406 receives and executes instructions. The data received by the system memory 1404 may optionally be stored on the removable storage 1408 or the non-removable storage 1410 before or after execution by the processing unit 1406.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Neuromuscular Electrical Stimulation

In one example implementation, NMES is used to improve CSF drainage in a non-invasive manner. In particular, transcutaneous electrical stimulation is applied to one or more of a subject's neck muscles, which include, but are not limited to, sternocleidomastoid, platysma, digastricus, and trapezoid muscles. Contraction of these muscles “squeezes” underlying lymph nodes improving lymph flow through the lymphatic vessels, which are endowed with intravascular valves preventing backflow, and hence improving unidirectional CSF drainage to the systemic blood circulation. NMES is applied in a particular sequence and at specific locations, which directs lymph flow away from the head.

For example, surface electrodes can be placed at the musculi sternocleidomastoideus, platysma and trapezius at their origin and at their insertions as shown in FIGS. 2A and 2B. Electrical stimulation is delivered in bursts of rectangular (0.5-1000 μsec, optionally 500 μsec) or sinusoidal pulses at 5-150 Hz (optionally 50-120 Hz). A series of 5-50 (optionally 10-20) bursts of 100-500 msec duration are delivered at frequency of 0.02-1 Hz (optionally 0.1-1 Hz). The current, determined by subjective reporting and muscle contraction, varies from 10-100 mA (optionally 30 to 100 mA). The series of bursts are repeated every 1 to 10 minutes for 15 minutes to one hour. For the treatment of persisting conditions which include, but are not limited to, hydrocephalus, Alzheimer's disease, and tauopathies, the stimulation can be repeated daily or with longer intervals depending on the desired effect or condition.

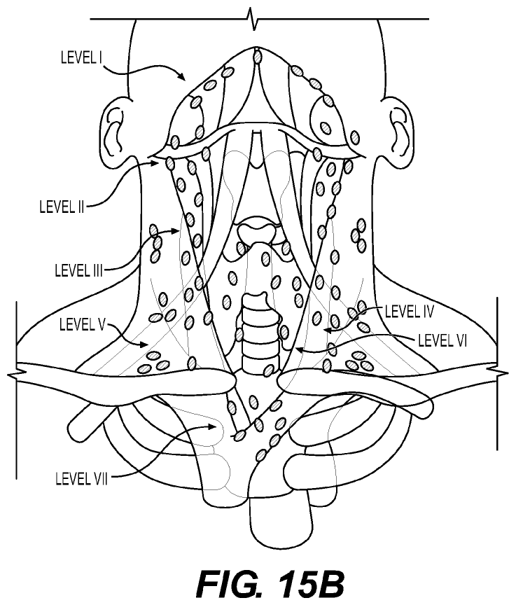

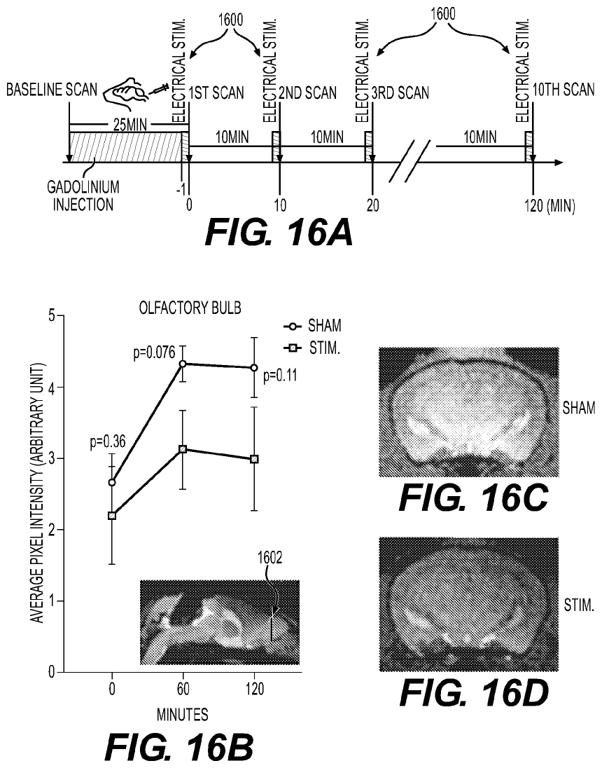

Referring now to FIGS. 16A-16D, example results illustrating accelerated CSF flow in response to NMES stimulation are shown. FIG. 16A is a schematic showing the time lapse of contrast-enhanced MRI experiments with the cisterna magna injection. In the experiments, the cisterna magna is catheterized and contrast agent is delivered thereto. Stimulation 1600 is applied as shown in FIG. 16A prior to MRI scans taken at 10 minute intervals for 2 hours. FIG. 16B shows quantification of gadolinium signal in the olfactory bulb. Average pixel intensity is quantified for a spherical region of interest (ROI) drawn in the olfactory bulb region, and normalized by the average pixel intensity in the muscle region. N=3 mice/group and p value calculated with multiple t-test and Holm-Sidak for multiple corrections. The insert shows the anatomical level of measurement 1602. FIG. 16C is a representative MRI coronal section for Sham mice (i.e., no stimulation) taken 60 minutes after the first scan. FIG. 16D is a representative MRI coronal section for Stimulated mice (0.2 Hz_5 Hz-Bottom) taken 60 minutes after the first scan. Comparing FIGS. 16C and 16D illustrates the accelerated CSF flow in response to NMES stimulation. In particular, the darker (less contrast) image in FIG. 16D demonstrates how the contrast agent drained with CSF in the stimulated mice.

### Mechanical Actuation

In another example implementation, mechanical actuators are used to improve CSF drainage in a non-invasive manner. In particular, mechanical actuators positioned (e.g. flexible rings around the neck but below the carotid bifurcation) around the neck or specifically over the neck's lymphatic nodes as shown in FIG. 5. The mechanical actuators are activated (e.g., inflated) to exert positive mechanical pressure over the nodes. Periodic actuation (e.g., inflation) is initiated in the most distal mechanical actuator followed by activation of the next more proximal actuator subsequently. The sequence of the actuator activation is such that the wave of lymphatic flow moves in proximal direction. Pressure is regulated from 2 to 25 mmHg and duration of each cycle in the single tube/actuator is regulated from 500 msec to 5 seconds. A series of sequential compressions (inflation or mechanical actuator) lasting in summary (including all tubes/actuators) for 5-15 min and repeated as necessary every 30 minutes. Each session can be repeated several time/day, several times/week depending on the condition.

### Vacuum Suction

In yet another example implementation, a vacuum suction device is used to improve CSF drainage in a non-invasive manner. In particular, a vacuum suction device is attached around the neck, or in separate segments on each side of the neck, but below the carotid bifurcation and positioned over the lymph nodes as shown in FIG. 8. The negative pressure inside the device is regulated to 3-5 mmHg, which when activated expands lymphatic nodes and thus suctioning lymph into the nodes. The suctions of 0.5-2 seconds duration can be repeated periodically at series of 2-10 suctions every 1 to 10 minutes. Treatment can be repeated for 5-30 minutes, several times a day, for time necessary to achieve an effective decrease of the hydrocephalus or levels of pathological substances in the cerebrospinal fluid.

### Implantable Balloon Device

In yet another example implementation, an implantable balloon device is used to improve CSF drainage. In particular, an expandable bubble is implanted at the junction of cisterna magna and perispinal subarachnoid space as shown in FIG. 11. The frontal wall of the bubble is thicker than the side and rear walls. The inflation of the implanted bubble compresses the cisterna magna in such a way that CSF is pushed toward the perispinal subarachnoid space, and further along the nerve roots and into the lymphatic system while diminishing retrograde flow to the fourth ventricle. The inflation occurs slowly (e.g., 1 mm/second) from 0 to 10-15 mmHg. The procedure can be repeated when necessary.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A system for draining cerebrospinal fluid (CSF) though a subject's neck lymphatic system, the system comprising:
a signal generator;
a plurality of electrodes operably connected to the signal generator; and
a controller operably connected to the signal generator, the controller comprising a processor and a memory, the memory having computer-executable instructions stored thereon that, when executed by the processor, cause the controller to:
deliver a neuromuscular electrical stimulation signal comprising at least one burst of pulses to at least one muscle in the subject's neck, wherein the neuromuscular electrical stimulation signal is configured to induce a plurality of contractions of the at least one muscle, wherein the contractions of the at least one muscle are configured to squeeze at least one lymph node to create a pumping force, and wherein the pumping force is configured to direct CSF flow in a proximal direction.

2. The system of claim 1, wherein the at least one burst of pulses comprises rectangular pulses.

3. The system of claim 2, wherein the rectangular pulses are monophasic or biphasic pulses.

4. The system of claim 2, wherein each of the rectangular pulses has a duration of about 0.5-1,000 microseconds (μsec).

5. The system of claim 1, wherein the at least one burst of pulses comprises sinusoidal pulses.

6. The system of claim 5, wherein the sinusoidal pulses have a frequency of about 5-150 Hertz (Hz).

7. The system of claim 1, wherein the neuromuscular electrical stimulation signal is delivered with a current of about 10-100 milliamps (mA).

8. The system of claim 1, further comprising a sensor configured to detect muscular contractions, wherein the memory has further computer-executable instructions stored thereon that, when executed by the processor, cause the controller to receive a feedback signal from the sensor, wherein the feedback signal comprises information related to a contraction state of the at least one muscle.

9. The system of claim 8, wherein the memory has further computer-executable instructions stored thereon that, when executed by the processor, cause the controller to increase a magnitude of the neuromuscular electrical stimulation signal until detecting a contraction of the at least one muscle.

10. The system of claim 9, wherein the memory has further computer-executable instructions stored thereon that, when executed by the processor, cause the controller to further increase the magnitude of the neuromuscular electrical stimulation signal above a minimum current that induces the contraction of the at least one muscle.

11. The system of claim 10, wherein the magnitude of the neuromuscular electrical stimulation signal current is increased about 10-50% above the minimum current.

12. The system of claim 8, wherein the sensor is a strain gauge.

13. The system of claim 1, wherein the neuromuscular electrical stimulation signal comprises a plurality of bursts of pulses.

14. The system of claim 13, wherein the plurality of bursts of pulses is a series of about 5-50 bursts of pulses.

15. The system of claim 14, wherein the series is delivered at a frequency of about 0.02-1 Hz.

16. The system of claim 1, wherein the neuromuscular electrical stimulation signal is delivered for a period of about 5-60 minutes.

17. The system of claim 1, wherein the at least one muscle is one or more of musculus platysma, sternocleidomastoid, or trapezius.

18. The system of claim 1, wherein the plurality of electrodes are surface electrodes.

19. The system of claim 1, wherein the plurality of electrodes are implantable electrodes.

20. The system of claim 19, wherein the signal generator is an implantable signal generator.

21-81. (canceled)
